(19) United States
(12) Patent Application Publication
(10) Pub. No.: US 2025/0049677 A1
(43) Pub. Date: Feb. 13, 2025

(54) Oxa-Macrocyclic Musk as Fragrance Compounds

(71) Applicant: Symrise AG, Holzminden (DE)

(72) Inventors: Vijayanand CHANDRASEKARAN, Frankenthal (DE); Bernd HÖLSCHER, Hameln (DE)

(21) Appl. No.: 18/718,894

(22) PCT Filed: Dec. 17, 2021
(86) PCT No.: PCT/EP2021/086566

§ 371 Date: Jun. 12, 2024

(51) Int. Cl. A61K 8/49; A61Q 13/00; C07D 313/00; C07D 321/00

(52) U.S. Cl. CPC A61K 8/4973; A61Q 13/00; C07D 313/00; C07D 321/00

(57) ABSTRACT

The invention is in the field of fragrances and relates to novel oxa-macrocyclic fragrance compounds according to general formula (I) with a musk-based note and improved biological degradability and further with improved chemical stabilities. The invention also relates to methods for preparing these compounds. Furthermore, the present invention also relates to fragrance compositions comprising one or more of the inventive compounds. Moreover, the invention relates to the use of these compounds or fragrance compositions as an odorant or for improving the fixation of a fragrance compound or a fragrance composition as well as for the preparation of a perfumed product. In addition, the present invention thus also refers to the use of said compounds or compositions for the preparation of a perfumed products as well as perfumed products as such.

### FIELD OF THE INVENTION

The invention is in the field of fragrances and relates to novel oxa-macrocyclic fragrance compounds according to general formula (I) with a musk-based note and improved biological degradability and further with improved stabilities. The invention also relates to methods for preparing these compounds. Furthermore, the present invention relates to fragrance compositions comprising one or more of the inventive compounds. Moreover, the invention relates to the use of these compounds or fragrance compositions as an odorant or for improving the fixation of a fragrance compound or a fragrance composition as well as for the preparation of a perfumed product. In addition, the present invention refers to the use of said compounds or compositions for the preparation of a perfumed products as well as perfumed products as such.

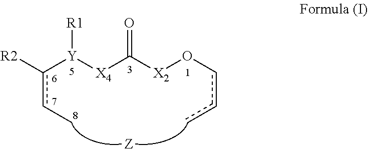

### PRIOR ART

Musk is a class of aromatic substances established as base notes in perfumery. They include glandular secretions from animals such as the musk deer, numerous plants emitting similar fragrances, and artificial substances with similar odours. Musk was a name originally given to a substance with a strong odour obtained from a gland of the musk deer. The substance has been used as a popular perfume fixative since ancient times and is one of the most expensive animal products in the world. It is applied to various plants and animals of similar smell (e.g. muskox) and has come to encompass a wide variety of aromatic substances with similar odours, despite their often differing chemical structures and molecular shapes.

A known organic compound with a characteristic odour of musk is Muscone, which consists of a 15-membered ring ketone with one methyl substituent in the 3-position. It is an oily liquid that is found naturally as the (−)-enantiomer, (R)-3-methylcyclopentadecanone. As a disadvantage, Muscone is only very slightly soluble in water and badly biodegradable.

However, the quest for synthetic substances having intense musk smelling properties and being both, chemically stable and biodegradable, is very high.

In particular, sufficient chemical stability is required in order to guarantee the stability of the fragrance material within product formulations and thus of the perfumed product as such and in turn to ensure a high product quality with consistent odour (i.e. high odour-stability). Degradation of the fragrance materials might cause undesired off-notes, a change in colour and the like.

Moreover, in terms of sustainability, there is an increasing demand for chemical compounds and in particular for fragrances having significantly improved biodegradability. Enhanced water-solubility of the compounds is therefore an enormous advantage.

The primary object of the present invention is thus to provide new musk fragrance compounds which exhibit superb odour characteristics, while simultaneously being chemically stable and biodegradable.

It has now been surprisingly found that the novel oxa-macrocyclic compounds according to general formula (I) comprising have a pronounced natural, balanced and intense musk odour as well as a high stability and enhanced tenacity, thus providing for excellent fragrance materials suitable for a broad range of applications. Moreover, the novel compounds according to general formula (I) have improved water-solubilities and are thus better biodegradable.

### SUMMARY OF THE INVENTION

In a first aspect, the present invention relates to a compound of general formula (I):

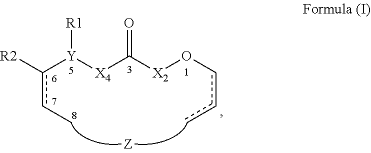

wherein in the general formula (I)
X represents methylene or ethylene, optionally substituted with a methyl group;
Y represents methylene or O;
R1 represents H, a methyl group or an ethyl group;
R2 represents H, a methyl group or an ethyl group;
Z represents an optional substituted C2, C3, C4, C5, C6 or C7 alkyl group; and
   optionally wherein at at least one position of the dotted lines there is a C═C double bond;
   or its stereoisomers, in particular enantiomers.

In a second aspect, the present invention relates to a fragrance composition comprising at least one compound according to the invention and at least one further fragrance substance.

In a third aspect, the present invention relates to a method for producing a compound of general formula (I) according to the invention. The method comprises the steps of:
(1) Preparing an olefin according to the general formula (II):

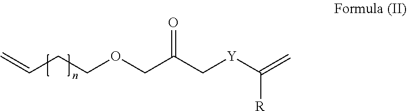

wherein n=7 or n=8;
wherein R represents H, methyl or ethyl;
wherein Y represents methylene or O and
2) olefin metathesis of the olefin prepared in step (1) to yield the compound of general formula (I).

In a fourth aspect, the present invention relates to the use of a compound according to the invention or the use of a fragrance composition comprising a compound according to the invention as an odorant or fragrance compound or for improving the fixation of a fragrance compound or a composition comprising a fragrance compound or for the preparation of a perfumed product.

In a further aspect, the present invention relates to a perfumed product comprising a compound according to the invention or the fragrance composition comprising a compound according to the invention.

In yet another aspect, the present invention relates to a perfumed product comprising a compound according to the invention or the fragrance composition comprising a compound according to the invention in an effective amount, and a carrier or substrate.

Finally, the present invention relates to a perfumed product as defined comprising a compound according to the invention or the fragrance composition comprising a compound according to the invention, wherein the perfumed product is a perfume oil, perfume base, formulation for personal hygiene, cleaning agent or air freshener.

The inventors have now established that the novel compound according to general formula (I) have a pronounced musk odour, partly with creamy, animalic and powdery notes along with high tenacities. Moreover, the compounds according to the invention show an improved chemical stability and improved water-solubility and are hence improved in terms of their biodegradability.

### DETAILED DESCRIPTION OF THE INVENTION

In accordance with the invention, the above object is achieved by an oxa-macrocyclic compound of general formula (I):

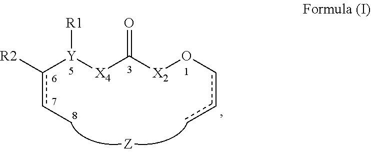

wherein in the general Formula (I):
X represents methylene or ethylene, optionally substituted with a methyl group;
Y represents methylene or O;
R1 represents H, a methyl group (CH₃) or an ethyl group;
R2 represents H, a methyl group or an ethyl group;
Z represents an optional substituted C2, C3, C4, C5, C6 or C7 alkyl group; and
   optionally wherein at at least one position of the dotted lines there is a C═C double bond;
   or its stereoisomers, in particular enantiomers.

The compounds of general formula (I) have an outstanding, balanced and characteristic musk odour profile with animalic, creamy, powdery and ergon notes as well as remarkable tenacities. Based on the additional creamy and powdery nuances, the presently developed compounds are suitable for the use as alternative fragrance materials with enhanced secondary properties and thus more natural odour profiles adding creamy and powdery accords which can be advantageously incorporated into a wide range of formulations to create a more vibrant scent. Thus, the present invention affords creamy and powdery notes together with animalic and musk notes.

Moreover, the oxa-macrocyclic compounds described herein further comprising the Ketone unexpectedly have both, a strong musk odour and a significantly improved water-solubility while being chemically stable. Therefore, these compounds are highly suitable as fragrance or in fragrance mixtures and for their use in preparations comprising these fragrances or fragrance mixtures, the fragrances or fragrance mixtures being better biodegradable, meaning more sustainable.

It has been found that such compounds according to the invention, wherein in the general formula (I):
X represents methylene;
Y represents methylene or O;
R1 represents H or a methyl group;
R2 represents H or a methyl group; and
the compound has at least one C═C double bond at any of the positions with the dotted lines showed unexpectedly a very intense musk odour while being highly soluble in water and chemically stable.

In general, it is clear to the skilled artisan, R1 being void in case Y represents O.

The inventors found that adherence on laundry depends on the ring size of the oxa-macrocyclic compound according to the invention.

Thus, it was surprisingly found that compounds according to formula (I) being a 15-membered ring or a 16-membered ring have significantly improved solubilities in water combined with improved adherence properties on laundry while still containing intense musk odours.

Thus, the compound according to general formula (I) is preferably a 13- to 18-membered ring, more preferably a 14 to 17, in particular a 15- or 16-membered ring.

As the ring size of the oxa-macrocyclic compound according to general formula (I) relates to the optional substituted C2, C3, C4, C5, C6 or C7 alkyl group represented by Z, the compound according to general formula (I) is a 14-membered ring and Z represents an optionally substituted C3 alkyl group; or the compound according to general formula (I) is a 15-membered ring and Z represents an optionally substituted C4 alkyl group; or the compound according to general formula (I) is a 16-membered ring and Z represents an optionally substituted C5 alkyl group; or the compound according to general formula (I) is a 17-membered ring and Z represents an optionally substituted C6 alkyl group; or the compound according to general formula (I) is a 18-membered ring and Z represents an optionally substituted C7 alkyl group.

In a preferred variant, the compound of general formula (I) according to the invention comprises a C═C double bond at the position of the other dotted lines indicated in the molecule.

The dotted lines are the most concise way of expressing these options in the structural formula. The meaning is also evident to the skilled person and embodiments thereof can be identified from the examples.

However, the best combination of water-solubility properties, chemical stabilities and odour profiles were achieved for embodiments of formula (I), wherein R2 represents H.

Surprisingly, improved water-solubility and odour profile was found for embodiments of formula (I), wherein R2 represents H and wherein X represents methylene.

However, the best combination of water-solubility properties, chemical stabilities and odour profiles was achieved for an embodiment of formula (I), wherein X represents methylene, wherein Y represents methylene, wherein R2 represents H, wherein Z represents a C4 alkyl group and wherein the C═C double bond is at position 6 and 7.

In particular, a preferred embodiment according to the invention is 1-oxacyclopentadec-6-en-3-one (A).

In an alternative variant, excellent water-solubility properties, chemical stabilities and odour profile was achieved for an embodiment of formula (I), wherein X represents methylene, wherein Y represents methylene, wherein R2 represents H, wherein Z represents a C5 alkyl group and wherein the C═C double bond is at position 6 and 7.

In particular, another preferred embodiment according to the invention is 1-oxacyclo-hexadec-6-en-3-one (B).

Both compounds, A and B each have an optimized and well-balanced ratio of hydrophilic and hydrophobic properties and hence improved biodegradability beside an intense musk odour.

Embodiments of the general formula (I), wherein R2 represents an alkyl group, preferably a methyl group or an ethyl group, have both, good solubilities in water and chemical stabilities. Moreover, those embodiments showed intense musk odours.

Thus, in an alternative variant, an embodiment of the general formula (I), wherein R2 represents a methyl group, shows distinct odour properties while still having good water-solubility and chemical stability.

In a preferred embodiment, excellent water-solubility properties, chemical stabilities and intense odour properties were achieved from compounds according to general formula (I), wherein X represents methylene, wherein Y represents methylene, wherein R2 represents a methyl group, wherein Z represents a C5 alkyl group, and wherein the C═C double bond is at position 6 and 7.

Hence, a further preferred embodiment according to the invention is 6-methyl-1-oxa-cyclohexadec-6-en-3-one (C).

Moreover, the inventors found out that embodiments of formula (I), wherein Y represents O, still have an intense odour profile and further an improved water-solubility.

Hence, alternative variants according to the invention with improved water-solubility properties are obtained by compounds of general formula (I), wherein X represents methylene, wherein Y represents O and wherein the compound comprises a C═C double bond at the position of the other dotted lines indicated in the molecule.

Preferably, a compound of general formula (I), wherein X represents ethylene, wherein Y represents O and wherein the compound comprises a C═C double bond at the position of the other dotted lines indicated in the molecule, in the compound, Z represents a C5 alkyl group.

Such 16-membered ring compounds have an improved solubility behaviour in water and high chemical resistance.

In particular, a further preferred embodiment according to the invention is 1,5-dioxacyclohexadec-14-en-3-one (D).

In order to obtain compounds according to general formula (I) that have an improved solubility behaviour in water and chemical resistance and further improved adherence properties on laundry, in the compounds, Z represents a C6 alkyl group.

Preferably, a compound of general formula (I), wherein X represents ethylene, wherein Y represents O and wherein the compound comprises a C═C double bond at the position of the other dotted lines indicated in the molecule, in the compound, Z represents a C6 alkyl group.

In particular, a further preferred embodiment according to the invention is 1,5-dioxacyclo-heptadec-15-en-3-one (E).

In a third aspect, the present invention relates to a method for producing a compound of general formula (I) according to the invention. The method comprises the steps of:
(1) Preparing an olefin according to the general formula (II):

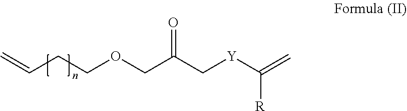

wherein n=7 or n=8;
   wherein R represents H, methyl or ethyl;
   wherein Y represents methylene or O; and
2) olefin metathesis of the olefin prepared in step (1) to yield the compound of general formula (I).

The method according to the invention provides simple and inexpensive access to the oxa-macrocyclic fragrance compounds according to general formula (I) with a musk-based note and improved biodegradability and further with improved chemical stabilities.

Preparation of olefins according to general formula (II) could be obtained starting from an allyl alcohol of the general formula (III):

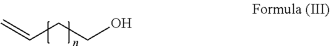

wherein n=7 or n=8.

In a first step, the allyl alcohol according to general formula (III) reacts with a halogenated acetic acid ester to give an ether.

In a second step, the ether obtained from the first step reacts with a substituted or non-substituted allylhalogenide to give the allylated ether compound.

And in a third step, the allylated ether compound obtained from the second step is decarboxylated.

Thus, olefins of general formula (II) are easily available from commercially available and inexpensive ally alcohols of general formula (III).

Alternatively, depending on the desired compound of general formula (I), olefins according to general formula (II) could be obtained starting from an allyl alcohol of the general formula (III):

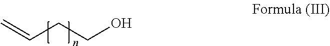

wherein n=7 or n=8.

In a first step, the allyl alcohol reacts to an oxirane.

In a second step, a ring opening reaction of the oxirane obtained from the first step is conducted with an allyl alcohol to give a tertiary alcohol.

In a third step, the tertiary alcohol obtained from the second step is oxidized.

Thus, olefins of general formula (II) are easily available from commercially available and inexpensive ally alcohols of general formula (III).

Olefin metathesis of the olefines according to general formula (II) could be realized by use of commercially available catalysts, e.g. Grubbs' catalysts.

Whenever reference is made in the present description to a compound of general formula (I), this is deemed to refer to all stereoisomers, in particular to all enantiomers, indifferently to the isomerically pure stereoisomers or mixtures of any of their stereoisomers. For economic reasons it is preferred to use the compounds as mixtures of their stereoisomers, in particular mixtures of their enantiomers.

The compounds of general formula (I) are thus present in the form of:
(a) a pure optically active enantiomer;
(b) a racemic mixture of the enantiomers, or
(c) an optically active mixture of various enantiomers.

Therefore, the present invention also discloses a mixture of any of the above compounds and/or a mixture of any of the above stereoisomers as well as the use of such compounds and/or mixtures in the meaning of the present application.

Generally, in the context of the present invention, the term “compounds of formula (I)” means both, the individual compounds of formula (I) (as well as their isomeric forms) and all mixtures of the compounds (and isomeric forms) of formula (I) in any mixing ratio. That is to say, statements in the following description concerning “compounds of formula (I)” apply both, to a single compound of formula (I), a single isomeric form and to mixtures consisting of or comprising compounds of formula (I) and their isomeric forms in any mixing ratio. Therefore, the present invention relates to the compounds according to the invention as such individually or also mixtures of the compounds according to the invention.

Moreover, with reference to general formula (I) and any C═C double bond contained therein, the compound is present in the form of geometric isomers, i.e. in Z and E configuration.

The compounds of general formula (I) according to the invention can either be used as individual substances or in mixtures with at least one other known fragrance substance selected from an extensive range of natural and synthetic substances available in a large number of fragrance mixtures and/or in admixture with one or more ingredients or excipients conventionally used in conjunction with odorants in fragrance compositions, for example carrier materials and other auxiliary agents commonly used in the art.

Therefore, in a second aspect, the present invention refers to a fragrance composition comprising at least one compound according to the invention and at least one further fragrance substance.

The following specified fragrance substances can be used, either as individual substances or in mixtures with at least one, two, three or even more fragrance substances, in a large number of fragrance mixtures, selected from an extensive range of natural and synthetic substances.

Fragrance substances which are advantageously suitable for combining are listed for example in S. Arctander, Perfume and Flavor Materials, volumes I and II, Montclair, N.J. 1969, private publication, and/or in H. Surburg, J. Panten, Common Fragrance and Flavor Materials, 6^{th} edition, Wiley-VCH, Weinheim 2016. The following list comprises examples of known odorant substances which are advantageously suitable for combining with the inventive compounds and mixtures thereof:
extracts of natural raw materials such as essential oils, concretes, absolutes, resins, resinoids, balsams, tinctures such as for example: ambergris tincture; amyris oil; angelica seed oil; angelica root oil; anise oil; valerian oil; basil oil; tree moss absolute; bay oil; artemisia oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; eucalyptus citriodora oil; eucalyptus oil; fennel oil; pine-needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; Litsea cubeba oil; bay leaf oil; mace oil; marjoram oil; mandarin oil; massoia bark oil; mimosa absolute; ambrette oil; musk tincture; muscatel sage oil; nutmeg oil; myrrh absolute; myrrh oil; myrtle oil; clove leaf oil; clove bud oil; neroli oil; olibanum absolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea tree oil; turpentine oil; thyme oil; Tolu balsam; tonka absolute; tuberose absolute; vanilla extract; violet leaf absolute; verbena oil; vetiver oil; juniper berry oil; cognac oil; wormwood oil; wintergreen oil; ylang ylang oil; hyssop oil; civet absolute; cinnamon leaf oil; cinnamon bark oil, and fractions thereof or constituents isolated therefrom;
individual fragrance substances from the group comprising hydrocarbons, such as for example: 3-carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
aliphatic alcohols, such as for example: hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixtures of 3,4,5,6,6-pentamethyl-3,4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and the acetals thereof, such as for example: hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citro-nellyloxyacetaldehyde; 1-(1-methoxypropoxy)-(E/Z)-3-hexene;
aliphatic ketones and the oximes thereof, such as for example: 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds, such as for example: 3-methylthio-hexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
aliphatic nitriles, such as for example: 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;
esters of aliphatic carboxylic acids, such as for example: (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl-isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; me-thyl-3,7-dimethyl-2,6-octadienoate; 4-methyl-2-pentyl-crotonate;
acyclic terpene alcohols, such as for example: citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
acyclic terpene aldehydes and ketones, such as for example: geranial; neral; citronellal; 7-hydroxy-3,7-dimethyloctanal; 7-methoxy-3,7-dimethyloctanal; 2,6,10-tri-methyl-9-undecenal; geranyl acetone; and the dimethyl and diethyl acetals of geranial, neral, 7-hydroxy-3,7-dimethyloctanal;
cyclic terpene alcohols, such as for example: menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;
cyclic terpene aldehydes and ketones, such as for example: menthone; isomenthone; 8-mercaptomenthan-3-one; carvone; camphor; fenchone; alpha-ionone; beta-ionone; alpha-n-methyl ionone; beta-n-methyl ionone; alpha-isomethyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; 2-methyl-4-(2,6,6-trimethyl-1-cyclohexen-1-yl)-2-butenal; nootkatone; dihydrono-otkatone; 4,6,8-megastigmatrien-3-one; alpha-sinensal; beta-sinensal; acetylated cedarwood oil (methylcedryl ketone);
cyclic alcohols, such as for example: 4-tert-butylcyclohexanol; 3,3,5-trimethyl-cyclo-hexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols, such as for example: alpha-3,3-trimethylcyclohexylmethanol; 1-(4-isopropylcyclohexyl)ethanol; 2-methyl-4-(2,2,3-trimethyl-3-cyclopent-1-yl)buta-nol; 2-methyl-4-(2,2,3-trimethyl-3-cyclopent-1-yl)-2-buten-1-ol; 2-ethyl-4-(2,2,3-trime-thyl-3-cyclopent-1-yl)-2-buten-1-ol; 3-methyl-5-(2,2,3-trimethyl-3-cyclopent-1-yl)-pen-tan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers, such as for example: cineole; cedryl methyl ether; cyclododecyl methyl ether; 1,1-dimethoxycyclododecane; (ethoxymethoxy)-cyclododecane; alpha-cedrene epoxide; 3a,6,6,9a-tetramethyldodecahydro-naphtho[2,1-b]furan; 3a-ethyl-6,6,9a-trimethyldodeca-hydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones, such as for example: 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic aldehydes such as for example: 2,4-dimethyl-3-cyclohexene carbaldehyde; 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde;
cycloaliphatic ketones, such as for example: 1-(3,3-dimethyl-cyclohexyl)-4-penten-1-one; 2,2-dimethyl-1-(2,4-dimethyl-3-cyclohexene-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl-(2,4-dimethyl-3-cyclohexen-1-yl)ketone;
esters of cyclic alcohols, such as for example: 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclo-pentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanoocta-hydro-5- or 6-indenyl acetate;
esters of cycloaliphatic alcohols, such as for example: 1-cyclohexylethyl crotonate; esters of cycloaliphatic carboxylic acids such as for example: allyl-3-cyclohexyl propionate; allylcyclohexyl oxyacetate; cis- and trans-methyl dihydrojasmonate; cis- and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolane 2-acetate;
araliphatic alcohols, such as for example: benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-di-methyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-pro-pen-1-ol; 4-methoxybenzyl alcohol; 1-(4-isopropylphenyl)ethanol;
esters of araliphatic alcohols and aliphatic carboxylic acids, such as for example: benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenyl-ethyl butyrate; cinnamyl acetate; 2-phenoxyethyl isobutyrate; 4-methoxybenzyl acetate; araliphatic ethers such as for example: 2-phenyl ethyl methyl ether; 2-phenyl ethyl isoamyl ether; 2-phenyl ethyl 1-ethoxyethyl ether; phenylacetaldehyde dimethylacetal; phenylacetaldehyde diethylacetal; hydratropaldehyde dimethylacetal; phenylacetaldehyde glycerol acetal; 2,4,6-trimethyl-4-phenyl-1,3-dioxane; 4,4a,5,9b-tetrahydro-indeno[1,2-d]-m-dioxin; 4,4a,5,9b-tetrahydro-2,4-dimethylindeno[1,2-d]-m-dioxin; aromatic and araliphatic aldehydes, such as for example: benzaldehyde; phenylacetaldehyde; 3-phenylpropanal; hydratropaldehyde; 4-methylbenz-aldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dim-ethoxybenzaldehyde; 2-methyl-3-(4-methoxyphenyl)propanal; 2-methyl-3-(4-methy-lenedioxyphenyl)propanal;
aromatic and araliphatic ketones, such as for example: acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3, 3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]-ethanone; 5′,6′,7′,8′-tetrahydro-3′,5′,5′,6′,8′,8′-hexamethyl-2-acetonaphthone;
aromatic and araliphatic carboxylic acids and the esters thereof, such as for example: benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethylbenzoate; ethyl-3-phenyl glycidate; ethyl-3-methyl-3-phenyl glycidate;
nitrogenous aromatic compounds, such as for example: 2,4,6-trinitro-1,3-dimethyl-5-tert-butylbenzene; 3,5-dinitro-2,6-dimethyl-4-tert-butyl aceto-phenone; cinnamonitrile; 3-methyl-5-phenyl-2-pentenoic acid nitrile; 3-methyl-5-phenylpentanoic acid nitrile; methyl anthranilate; methyl-N-methyl anthranilate; Schiff bases of methyl anthranilate with 7-hydroxy-3,7-dimethyloctanal, 2-methyl-3-(4-tert-butylphenyl)propanal or 2,4-dimethyl-3-cyclohexene carbaldehyde 6-isopropyl quinoline; 6-isobutyl quinoline; 6-sec-butyl quinoline; 2-(3-phenylpropyl)pyridine; indole; skatole; 2-methoxy-3-iso-propylpyrazine; 2-isobutyl-3-methoxypyrazine;
phenols, phenyl ethers and phenyl esters, such as for example: estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl acetate;
heterocyclic compounds, such as for example: 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones, such as for example 1,4-octanolide; 3-methyl-1,4-octanolide; 1,4-nonanolide; 1,4-decanolide; 8-decen-1,4-olide; 1,4-undecanolide; 1,4-dodecan-olide; 1,5-decanolide; 1,5-dodecanolide; 4-methyl-1,4-decanolide; 1,15-penta-decanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin;
and mixtures of the above substances.

In such fragrance compositions, the compounds according to the present invention exhibit a positive influence on the overall fragrance chord by distinctly enhancing the musk nature of the final composition with animalic notes and simultaneously offering good tenacity, fixation and substantivity. The presently described compounds according to the invention thus can be used as efficient and highly stable fragrance compounds.

In fragrance compositions, the amount of the compound according to the invention used is preferably ranging from 0.0001 to 90% by weight, preferably 0.01 to 70% by weight and particularly preferably 0.1 to 50% by weight, relative to the total amount of the fragrance composition.

Fragrance compounds according to the invention and fragrance compositions which comprise or contain the at least one compound according to the invention may be used for perfuming applications in liquid form, undiluted or diluted with a solvent. Solvents suitable for this purpose are for example ethanol, isopropanol, diethylene glycol monoethyl ether, glycerol, propylene glycol, 1,2-butylene glycol, dipropylene glycol, diethyl phthalate, triethyl citrate, isopropyl myristate, triacetin, vegetable oils, etc.

Moreover, the fragrance compounds according to the invention or fragrance compositions which comprise or contain the compounds according to the invention may be adsorbed on a carrier which ensures both a fine distribution of the fragrance substances in the final product and controlled release upon use. Such carriers may be porous inorganic materials such as sodium sulphate, silica gels, zeolites, gypsums, clays, clay granules, aerated concrete etc., or organic materials such as woods, cellulose-based substances, sugars, dextrins (for example maltodextrin), or plastics such as PVC, polyvinyl acetates or polyurethanes.

Fragrance compounds according to the invention and fragrance compositions which comprise or contain the compound(s) according to the invention indicated by formula (I) may also be microencapsulated, spray-dried, be provided as inclusion complexes or as extrusion products (i.e. products according to the invention).

Optionally, the properties of the fragrance compounds or compositions modified in such a way may be further optimised with regard to a more targeted fragrance release by “coating” with suitable materials, for which purpose waxy plastics, such as for example polyvinyl alcohol, are preferably used. The resultant products are in turn products according to the invention.

The fragrance compounds according to the invention or fragrance compositions according to the invention may be encapsulated, for example, by coacervation methods with the assistance of capsule materials made, for example, from poly-urethane-type substances or soft gelatine.

Spray-dried fragrance formulations based on fragrance compounds according to the invention and fragrance compositions which comprise or contain the compound(s) according to the invention may be produced for example by spray-drying an emulsion or dispersion comprising or containing the fragrance compound or composition, wherein modified starches, proteins, dextrin and vegetable gums may be used as carriers. Inclusion complexes may be produced for example by introducing dispersions of the fragrance compound or composition and cyclodextrins or urea derivatives into a suitable solvent, for example water.

Extrusion products may be produced by melting the fragrance compound or compositions with a suitable waxy substance and extruding with subsequent solidification, optionally in a suitable solvent, for example isopropanol.

In another aspect, the present invention thus relates to the use of a compound according to general formula (I) or a fragrance composition comprising or containing a compound according to the invention as an efficient odorant or fragrance compound. The comments made above apply correspondingly to preferred compounds and mixtures.

The present invention also relates to the use of a compound according to general formula (I) or a fragrance composition comprising or containing at least a compound according to the invention for improving the fixation of a fragrance compound while making simultaneous use of the compound or of the composition comprising or containing the compound according to the invention as a fragrance substance and for preparing perfumed products. With regard to the preferred choice of compounds and mixtures according to the invention, the comments made above of course apply correspondingly.

Therefore, the present invention relates to the use of said compounds and compositions for improving the fixation of a fragrance compound or for the preparation of a perfumed product.

The compounds or compositions which comprise or contain a compound according to the invention may be incorporated into products which are perfumed or are intended to be perfumed, in particular formulations serving for personal hygiene such as perfume oils, perfume bases, formulations for personal hygiene, cleaning agents or air fresheners.

Thus, in another aspect, the present invention also refers to perfumed products comprising or containing a compound according to formula (I) or a fragrance composition comprising or containing at least one compound according to the invention.

In accordance with a preferred variant, the perfumed products according to the invention comprising or containing the compound or a fragrance composition which comprises or contains a compound according to the invention are produced by incorporating the compound or composition which comprises or contains a compound according to the invention, in pure form without a solvent, as a solution or in the form of a mixture with a solid or liquid carrier and optionally other auxiliaries and/or stabilisers to form a base preparation serving for personal hygiene such as cleaning agents and the like.

The present invention therefore also provides a perfumed product comprising or containing a compound of general formula (I) or a fragrance composition which comprises or contains at least a compound according to the invention in an effective amount in combination with a carrier or substrate.

A sensory effective amount means that already a proportion of the substances according to general formula (I) in the mixture is sufficient to emanate a pleasant perceptible fragrance impression, especially a musk odour impression. This perceive-impression is generally achieved when at least 0.001 wt. % of the compounds according to general formula (I), is present in the final preparation/formulation.

The compounds of formula (I) described herein have, in addition to their intensive sensory properties, additional positive secondary properties, such as a high stability under certain application conditions. This stability, especially in acidic media qualifies the inventive compounds for use and processing in a wide range of applications.

Perfumed products according to the invention are for example perfume extracts, eaux de parfum, eaux de toilette, shaving lotions, eaux de cologne, pre-shave products, splash colognes and perfumed tissue wipes, including for perfuming acidic, alkaline and neutral cleaning agents such as for example floor cleaners, window cleaners, dishwashing detergents, bath and sanitary cleaners, scouring creams, solid and liquid toilet cleaners, pulverulent and foam carpet cleaners, textile fresheners, ironing aids, liquid detergents, pulverulent detergents, laundry pre-treatment agents such as bleaches, soaking agents and stain removers, laundry rinse conditioners, laundry soaps, laundry tablets, disinfectants, surface disinfectants as well as air fresheners in liquid or gel form or those applied to a solid carrier, aerosol sprays, waxes and polishes such as furniture polishes, floor waxes and shoe polishes as well as body care products such as for example solid and liquid soaps, shower gels, shampoos, shaving soaps, shaving foams, bath oils, cosmetic emulsions of the oil-in-water, water-in-oil and water-in-oil-in-water type such as for example skin creams and lotions, face creams and lotions, sunscreen creams and lotions, after-sun creams and lotions, hand creams and lotions, foot creams and lotions, depilatory creams and lotions, after-shave creams and lotions, tanning creams and lotions, hair care products such as for example hair sprays, hair gels, strengthening hair lotions, hair rinses, permanent and semi-permanent hair dyes, hair styling agents such as cold waving and hair straightening agents, hair tonics, hair creams and lotions, deodorants and antiperspirants such as for example underarm sprays, roll-ons, deodorant sticks, deodorant creams, decorative cosmetic products such as for example eyeshadow, nail varnish, make-up products, lipstick, mascara, as well as candles, lamp oils, incense sticks, insecticides, repellents and fuels or oral and/or dental care products such as toothpastes, tooth gels, tooth powders, mouthwashes, chewing gum and other oral care products.

The products according to the invention may also be semi-finished products comprising a compound of general formula (I) or a fragrance composition which contains or comprises at least a compound according to the invention in an effective amount.

The percentages at which the compound of general formula (I) or a fragrance composition which contains or comprises at least a compound according to the invention is used may vary within wide limits, ranging from a few parts per thousand in mass market products such as cleaning agents up to a few percent in alcoholic extracts for fine perfumery. However, even small amounts of the novel compounds or a fragrance composition which comprises or contains at least a compound according to the invention efficiently provide a rich sandalwood or woody note to the perfumed formulation/product. Preferably, the compound of general formula (I) or a fragrance composition which comprises or contains at least a compound according to the invention is used in an amount of 0.0001 to 90% by weight, preferably 0.01 to 70% by weight and particularly preferably 0.1 to 50% by weight, based on the total weight of the final (product) formulation.

Finally the present invention relates to perfumed products, wherein the perfumed product is preferably a perfume oil, perfume base, formulation for personal hygiene, cleaning agent or air freshener.

### EXAMPLES

The following compounds were prepared according the methods indicated below. The structure determination by means of spectroscopy, was carried out by known techniques.

As indicated above, all compounds disclosed herein as well as all formulas generally should encompass both, the pure stereoisomers, in particular enantiomers, and mixtures of the stereoisomers, in particular mixtures of the enantiomers. Also, all intermediates obtained from the process can occur in different isomeric forms, and, thus, should encompass both the pure stereoisomers and mixtures of the stereoisomers.

### Example 1: Preparation of 1-oxacyclopentadec-6-en-3-one (A)

### Step 1: Preparation of 2 ethyl 4-dec-9-enoxy-3-oxo-butanoate

To a mixture of 9-decen-1-ol (0.64 mol) in dry THF and DMF was added NaH (1.28 mol) in several portions at 20° C. under N₂. The mixture was stirred at 20° C. for 1 h, then ethyl 4-chloroacetoacetonate (0.77 mol) was added dropwise at 20° C. and the mixture was stirred at 25° C. overnight. The reaction mixture was quenched with H₂O and extracted with EtOAc. The combined organic layers were washed with brine, dried over Na₂SO₄ and concentrated on vacuo at 45° C. to afford the crude product, which was purified by column chromatography to give 2 ethyl 4-dec-9-enoxy-3-oxo-butanoate.

¹H-NMR: (400 MHz, CDCl₃) δ 1.19-1.23 (m, 11H), 1.47-1.56 (m, 4H), 1.94-1.99 (m, 2H), 3.40-3.45 (m, 4H), 4.01 (s, 2H), 4.09-4.15 (m, 2H), 4.84-4.94 (m, 2H), 5.70-5.77 (m, 1H).

### Step 2: Preparation of ethyl 2-(2-dec-9-enoxyacetyl)pent-4-enoate

To a mixture of 2 ethyl 4-dec-9-enoxy-3-oxo-butanoate (0.56 mol) in dry EtOH EtONa (2.25 mol) was added in several portions at 20° C. under N₂. The mixture was stirred at 20° C. for 1 h, then 3-bromoprop-1-ene (1.12 mol) was added dropwise and the mixture was stirred at 80° C. overnight. The reaction mixture was quenched with H₂O, concentrated to remove EtOH and the aqueous layer was extracted with EtOAc. The combined organic layers were washed with brine, dried over Na₂SO₄ and concentrated on vacuo at 45° C. to afford the crude product, which was purified by column chromatography to give ethyl 2-(2-dec-9-enoxyacetyl)pent-4-enoate.

¹H-NMR: (400 MHz, CDCl₃) δ 1.23-1.36 (m, 13H), 1.56-1.60 (m, 2H), 2.02-2.05 (m, 2H), 2.57-2.63 (m, 2H), 3.45 (t, J=6.8 Hz, 2H), 3.71 (t, J=7.2 Hz, 1H), 4.09-4.19 (m, 4H), 4.90-5.11 (m, 4H), 5.74-5.83 (m, 2H).

### Step 3: Preparation of 1-dec-9-enoxyhex-5-en-2-one

To a mixture of ethyl 2-(2-dec-9-enoxyacetyl)pent-4-enoate (0.092 mol) in DMSO and H₂O, LiCl (0.18 mol) was added in one portion at 20° C. under N₂, then the mixture was stirred at 200° C. overnight. The reaction mixture was quenched with H₂O and extracted with EtOAc. The combined organic layers were washed with brine, dried over Na₂SO₄ and concentrated on vacuo at 45° C. to afford the crude product, which was purified by column chromatography to give 1-dec-9-enoxyhex-5-en-2-one.

¹H-NMR: (400 MHz, CDCl₃) δ 1.24-1.36 (m, 10H), 1.57-1.62 (m, 2H), 2.00-2.03 (m, 2H), 2.32-2.34 (m, 2H), 2.54-2.58 (m, 2H), 3.44-3.47 (m, 2H), 4.00 (s, 2H), 4.90-5.05 (m, 4H), 5.76-5.83 (m, 2H).

### Preparation of 1-oxacyclopentadec-6-en-3-one (A)

The mixture of 1-dec-9-enoxyhex-5-en-2-one (0.039 mol), Hoveyda-Grubbs 2^{nd} in dry DCM was stirred at 45° C. overnight under Ar. The reaction mixture was concentrated on vacuo at 40° C. to afford the crude product, which was purified by column chromatography followed by recrystallization and prep-HPLC to give 1-oxacyclopentadec-6-en-3-one (A).

¹H-NMR: (400 MHz, CDCl₃) δ 1.25-1.34 (m, 8H), 1.43-1.45 (m, 2H), 1.53-1.57 (m, 2H), 2.01-2.04 (m, 2H), 2.34-2.36 (m, 2H), 2.53-2.56 (m, 2H), 3.55-3.58 (t, J=5.6 Hz, 2H), 4.02 (s, 2H), 5.37-5.46 (m, 2H).

Odor: Creamy, animalic and musk

### Example 2: Preparation of 1-oxacyclohexadec-6-en-3-one (B)

### Step 1: Preparation of ethyl 3-oxo-4-undec-10-enoxy-butanoate

A mixture of Compound 10-undecen-1-ol (0.58 mol) in dry THF and DMF, NaH (1.17 mol) was added in several portions at 20° C. under N₂. The mixture was stirred at 20° C. for 1 h, then ethyl 4-chloroacetoacetonate (0.70 mol) was added at 20° C. and the mixture was stirred at 25° C. overnight. The reaction mixture was quenched with H₂O and extracted with EtOAc. The combined organic layers were washed with brine, dried over Na₂SO₄ and concentrated on vacuo at 45° C. to afford the crude product, which was purified by column chromatography to give ethyl 3-oxo-4-undec-10-enoxy-butanoate.

¹H-NMR: (400 MHz, CDCl₃) δ 1.25-1.36 (m, 15H), 1.56-1.60 (m, 2H), 2.01-2.03 (m, 2H), 3.45-3.48 (m, 2H), 3.51 (s, 2H), 4.07 (s, 2H), 4.15-4.21 (m, 2H), 4.90-4.99 (m, 2H), 5.76-5.83 (m, 1H).

### Step 2: Preparation of ethyl 2-(2-undec-10-enoxyacetyl)pent-4-enoate

To a mixture of ethyl 2-(2-undec-10-enoxyacetyl)pent-4-enoate (0.33 mol) in dry EtOH, EtONa was added (1.34 mol) in several portions at 20° C. under N₂. The mixture was stirred at 20° C. for 1 h, then 3-bromoprop-1-ene (0.67 mol) was added dropwise and the mixture was stirred at 80° C. overnight. The reaction mixture was quenched with H₂O, concentrated to remove EtOH and the aqueous layer was extracted with EtOAc. The combined organic layers were washed with brine, dried over Na₂SO₄ and concentrated on vacuo at 45° C. to afford the crude product, which was purified by column chromatography to give of ethyl 2-(2-undec-10-enoxyacetyl)pent-4-enoate.

¹H-NMR: (400 MHz, CDCl₃) δ 1.24-1.36 (m, 15H), 1.57-1.60 (m, 2H), 2.00-2.03 (m, 2H), 2.57-2.63 (m, 2H), 3.43-3.46 (m, 2H), 3.70 (t, J=7.2 Hz, 1H), 4.09-4.17 (m, 4H), 4.90-5.10 (m, 4H), 5.74-5.83 (m, 2H).

### Step 3: Preparation of 1-undec-10-enoxyhex-5-en-2-one

To a mixture of ethyl 2-(2-undec-10-enoxyacetyl)pent-4-enoate (0.088 mol) in DMSO and H₂O, LiCl (0.18 mol) was added in one portion at 20° C. under N₂ and the mixture was stirred at 200° C. overnight. The reaction mixture was quenched with H₂O and extracted with EtOAc. The combined organic layers were washed with brine, dried over Na₂SO₄ and concentrated on vacuo at 45° C. to afford the crude product, which was purified by column chromatography to give 1-undec-10-enoxyhex-5-en-2-one.

¹H-NMR: (400 MHz, CDCl₃) δ 1.27-1.36 (m, 12H), 1.56-1.62 (m, 2H), 2.00-2.03 (m, 2H), 2.33-2.36 (m, 2H), 2.54-2.58 (m, 2H), 3.44-3.47 (m, 2H), 4.02 (s, 2H), 4.90-5.05 (m, 4H), 5.76-5.85 (m, 2H)

### Preparation of (6)-1-oxacyclohexadec-6-en-3-one (B)

A mixture of 1-undec-10-enoxyhex-5-en-2-one (0.037 mol) and Hoveyda-Grubbs 2^{nd} in dry DCM was stirred at 45° C. overnight under Ar. The reaction mixture was concentrated on vacuo at 40° C. to afford the crude product, which was purified by column chromatography followed by recrystallization, distillation and prep-HPLC to give (6)-1-oxacyclohexadec-6-en-3-one (B).

¹H-NMR: (400 MHz, CDCl₃) δ 1.26-1.45 (m, 12H), 1.64 (m, 2H), 2.03 (m, 2H), 2.32 (m, 2H), 2.62-2.64 (m, 2H), 3.53 (m, 2H), 3.98 (s, 2H), 5.31-5.41 (m, 2H)

Odor: Musk, animalic

### Example 3: Preparation of 6-methyl-1-oxacyclohexadec-6-en-3-one (C)

6-methyl-1-oxacyclohexadec-6-en-3-one (C) was prepared analogous to 1-oxacyclopentadec-6-en-3-one (B), using 3-Bromo-2-methyl propene.

¹H-NMR (400 MHz, CDCl₃) δ 1.17-1.41 (m, 10H), 1.41-1.52 (m, 2H), 1.62 (s, 3H), 1.64 (t, J=6.4 Hz, 2H), 2.03 (q, J=6.8 Hz, 2H), 2.27 (t, J=7.4 Hz, 2H), 2.69 (t, J=7.4 Hz, 2H), 3.52 (t, J=5.5 Hz, 2H), 3.97 (s, 2H), 5.15 (tt, J=5.9, 1.4 Hz, 1H).

¹³C-NMR (101 MHz, CDCl₃) δ 16.4, 25.5, 26.8, 27.0, 27.4, 27.7, 28.0, 28.5, 29.2, 32.9, 38.1, 71.2, 76.4, 125.8, 133.8, 210.6.

Odor: Strong musk, ergon, powdery, creamy

### Example 4: Preparation of 1,5-dioxacyclohexadec-14-en-3-one (D)

### Step 1: Preparation of 2-(dec-9-enoxymethyl)oxirane

The mixture of NaOH (0.96 mol) and 9-decen-1-ol (0.32 mol) was stirred at 20° C. for 4 hrs. Then 2-(chloromethyl)oxirane (0.64 mol) was added and the mixture was stirred at 40° C. for 18 hrs. The mixture was then evaporated to give a crude product, which was purified by column chromatography to give 2-(dec-9-enoxy-methyl)oxirane.

¹H-NMR: (400 MHz, CDCl₃) δ 1.22-1.30 (m, 11H), 1.49-1.53 (m, 2H), 1.96-1.98 (m, 2H), 2.53-2.55 (m, 1H), 2.72 (t, J=4.4 Hz, 1H), 3.05-3.11 (m, 1H), 3.32-3.43 (m, 3H), 3.61-3.64 (m, 1H), 4.85-4.94 (m, 2H), 5.71-5.77 (m, 1H).

Step 2: Preparation of 1-allyloxy-3-dec-9-enoxy-propan-2-ol

To 2-Propene-1-ol (350 mL), Na (0.85 mol) was added portion wise under N₂. After the solution was stirred for 2 hrs, 2-(dec-9-enoxymethyl)oxirane (0.28 mol) was added to mixture and the mixture was stirred at 20° C. for 2 hrs. The mixture was evaporated to give a crude product, which was purified by column chromatography to give 1-allyloxy-3-dec-9-enoxy-propan-2-ol.

¹H-NMR: (400 MHz, CDCl₃) δ 1.18-1.32 (m, 12H), 1.45-1.48 (m, 2H), 1.94-1.99 (m, 2H), 3.37-3.44 (m, 6H), 3.88-3.96 (m, 3H), 4.84-4.94 (m, 2H), 5.11-5.23 (m, 2H), 5.73-5.87 (m, 2H).

### Step 3: Preparation of 1-allyloxy-3-dec-9-enoxy-propan-2-one

To a solution of 1-allyloxy-3-dec-9-enoxy-propan-2-ol (0.26 mol) in DCM, DMP (0.4 mol) was added at 0° C. and the mixture was stirred at 20° C. overnight. Then, the mixture was washed with brine and 5% NaHCO₃, dried over Na₂SO₄ and evaporated to give a crude product, which was purified by column chromatography to give 1-allyloxy-3-dec-9-enoxy-propan-2-one as a colourless oil.

¹H-NMR: (400 MHz, CDCl₃) δ 1.18-1.30 (m, 12H), 1.53-1.55 (m, 2H), 1.94-1.99 (m, 2H), 3.41 (t, J=6.8 Hz, 2H), 3.99 (d, J=5.6 Hz, 2H), 4.13 (s, 2H), 4.17 (s, 2H), 4.84-4.94 (m, 2H), 5.16-5.25 (m, 2H), 5.70-5.84 (m, 2H).

### Preparation of 1,5-dioxacyclohexadec-14-en-3-one (D)

The mixture of 1-allyloxy-3-dec-9-enoxy-propan-2-one (0.029 mol), Hoveyda-Grubbs 2^{nd} in dry DCM was stirred at 45° C. overnight under Ar. The reaction mixture was concentrated on vacuo at 40° C. to afford the crude product, which was purified by column chromatography followed by recrystallization and prep-HPLC to give 1,5-dioxacyclohexadec-14-en-3-one (D).

¹H-NMR: (400 MHz, CDCl₃) δ 1.25-1.47 (m, 12H), 2.04-2.07 (m, 2H), 3.39-3.43 (m, 2H), 3.94 (s, 2H), 4.02-4.03 (m, 2H), 4.35 (s, 2H), 5.36-5.43 (m, 1H), 5.54-5.60 (m, 1H)

### Example 5: Preparation of 1,5-dioxacycloheptadec-15-en-3-one (E)

1,5-dioxacycloheptadec-15-en-3-one (E) was prepared analogue to 1,5-dioxacyclohexadec-14-en-3-one (D) by starting from 10-undecen-1-ol.

¹H-NMR: (400 MHz, CDCl₃) δ 1.26-1.40 (m, H), 1.40-1.49 (m, 4H), 1.55-1.64 (m, 2H), 2.09-2.16 (m, 2H), 3.49 (dd, J=5.8, 4.9 Hz, 2H), 4.02 (s, 2H), 4.10 (dd, J=6.5, 1.1 Hz, 2H), 4.40 (s, 2H), 5.48 (dtt, J=15.6, 6.5, 1.4 Hz, 1H), 5.65 (dtt, J=15.1, 6.9, 1.3 Hz, 1H).

¹³C-NMR (101 MHz, CDCl₃) δ 26.6, 27.1, 27.8, 28.2, 28.5, 28.6, 29.0, 31.6, 71.4, 71.7, 72.1, 75.8, 126.5, 136.8, 206.6.

Odor: powdery, musky

1. An oxa-macrocyclic compound of general formula (I): 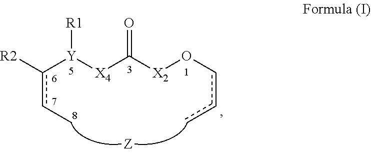
wherein in the general formula (I)
X represents methylene or ethylene, optionally substituted with a methyl group;
Y represents methylene or O;
R1 represents H, a methyl group or an ethyl group;
R2 represents H, a methyl group or an ethyl group;
Z represents an optionally substituted C2, C3, C4, C5, C6 or C7 alkyl group; and
wherein optionally for at least one position of the dotted lines there is a C═C double bond;
or its stereoisomers or enantiomers.

2. The compound according to claim 1, wherein
the compound is a 14-membered ring and Z represents an optionally substituted C3 alkyl group; or
wherein the compound is a 15-membered ring and Z represents an optionally substituted C4 alkyl group; or
wherein the compound is a 16-membered ring and Z represents an optionally substituted C5 alkyl group; or
wherein the compound is a 17-membered ring and Z represents an optionally substituted C6 alkyl group; or
wherein the compound is a 18-membered ring and Z represents an optionally substituted C7 alkyl group.

3. The compound according to claim 1, wherein in the general formula (I)
X represents methylene;
Y represents methylene or O;
R1 represents H or a methyl group;
R2 represents H or a methyl group; and
the compound has at least one C═C double bond at any of the positions with the dotted lines.

4. The compound according to claim 3, wherein the compound is a 15-membered or a 16-membered ring.

5. The compound according to claim 1, wherein the compound is present in the form of:
(a) a pure optically active enantiomer;
(b) a racemic mixture of the enantiomers; or
(c) an optically active mixture of various enantiomers.

6. The compound according to claim 1, wherein the compound of general Formula (I) is selected from the group consisting of the following compounds:

| Compound | |
|---|---|
| No. | Structure |
| A | 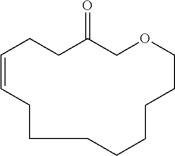 [SMILES: O=C1CC/C=C\CCCCCCCCOC1] |
| B | 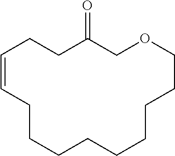 [SMILES: O=C1CC/C=C\CCCCCCCCCOC1] |
| C | 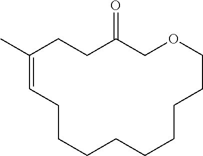 [SMILES: C/C1=C/CCCCCCCCCOCC(=O)CC1] |
| D | 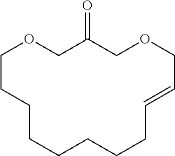 [SMILES: O=C1COC/C=C/CCCCCCCCOC1] |
| E | 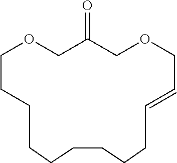 [SMILES: O=C1COC/C=C/CCCCCCCCCOC1] |

and their stereoisomers or enantiomers.

7. A fragrance composition comprising
(a) at least one compound according to claim 1; and optionally
(b) at least one further fragrance substance.

8. A method for producing a compound of general formula (I) according to claim 1, comprising the steps of:
(1) Preparing an olefin according to the general formula (II): 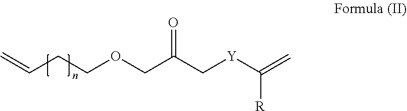
wherein n=7 or n=8;
wherein R represents H, methyl or ethyl;
wherein Y represents methylene or O and
2) performing olefin metathesis of the olefin prepared in step (1) to yield the compound of general formula (I).

9. The method according to claim 8,
wherein the olefin according to general formula (II) is prepared staring from an allyl alcohol of the general formula (III): 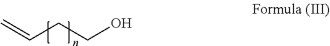
wherein n=7 or n=8;
wherein, in a first step, the allyl alcohol reacts with a halogenated acetic acid ester to give an ether;
wherein, in a second step, the ether obtained from the first step reacts with a substituted or non-substituted allylhalogenide to give an allylated ether compound and
wherein, in a third step, the allylated ether compound obtained from the second step is decarboxylated.

10. The method according to claim 8,
wherein the olefin according to general formula (II) is prepared staring from an allyl alcohol of the general formula (III): 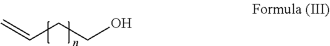
wherein n=7 or n=8;
wherein, in a first step, the allyl alcohol reacts to form an oxirane;
wherein, in a second step, a ring opening reaction of the oxirane obtained from the first step is conducted with an allylalcohol to give a tertiary alcohol;
wherein, in a third step, the tertiary alcohol obtained from the second step is oxidized.

11. A method of producing, enhancing or modifying a musk odor in a formulation
comprising the following steps:
providing a compound according to claim 1 or a composition comprising at least one compound according to claim 1 and optionally at least one further fragrance substance;
providing a composition with other constituents; and
mixing the composition with other constituents with an amount of the compound according to claim 1 or the composition comprising at least one compound according to claim 1 and optionally at least one further fragrance substance which is sufficient
(a) to produce a musk odor in the resultant complete mixture; or
(b) to enhance an existing musk odor in the composition with other constituents; or
(c) to modify an existing musk odor in the composition with other constituents.

12. A method of preparing a perfumed product comprising formulating the product with a compound according to claim 1 or a composition comprising at least one compound according to claim 1 and optionally at least one further fragrance substance as an odorant.

13. A method for improving the fixation of a fragrance compound in a perfumed product comprising formulating the product with a compound according claim 1 or a composition comprising at least one compound according to claim 1 and optionally at least one further fragrance substance.

14. A perfumed product comprising a compound according to claim 1 or a composition comprising at least one compound according to claim 1 and optionally at least one further fragrance substance in a sensory effective amount, and optionally comprising a carrier or substrate.

15. The perfumed product according to claim 14, wherein the perfumed product is a perfume oil, perfume base, formulation for personal hygiene, cleaning agent or laundry agent.
